(19) United States
(12) Patent Application Publication
(10) Pub. No.: US 2020/0197924 A1
(43) Pub. Date: Jun. 25, 2020

(54) DOSING DEVICE

(71) Applicant: BIOTECHNOLOGY INSTITUTE, I MAS D, S.L., Vitoria (Alava) (ES)

(72) Inventor: Eduardo ANITUA ALDECOA, Vitoria (Alava) (ES)

(21) Appl. No.: 16/711,611

(22) Filed: Dec. 12, 2019

(30) Foreign Application Priority Data
Dec. 21, 2018 (ES) P 201831270

(51) Int. Cl. B01L 3/02

(52) U.S. Cl. CPC B01L 3/0224; B01L 2200/16; B01L 2400/0475; B01L 2300/06; B01L 2300/0832

(57) ABSTRACT

Dosing device (1) for the dosage of blood compounds, medical treatments or other substances, comprising a stepped body (9) in a plunger (3) and a presser mechanism (17) in the tube (2) of a syringe. The projection (19) from the pusher (18) of the presser mechanism (17) transmits a downwards force (F) exerted by the user, onto the step (12) of the stepped body (9) by pushing it down a distance (x) with the plunger (3). In this way, the dosing device (1) can measure an exact dose of the compound manually, quickly and easily without the need for additional measurements.

### TECHNICAL FIELD

The invention relates to a device for the manual dosage of blood compounds, medical treatments or other fluid compounds used in the field of medicine and dentistry.

### STATE OF THE ART

In the field of medicine and dentistry, syringes and dosing devices are known to be used; they are needed to create compounds of different substances, prepare blood compounds or provide certain treatments, among many other uses and applications.

For the manual dosage of compounds, treatments or substances, traditional syringes are generally used, based on a scale printed on a cylindrical tube, which is normally transparent, so that when a plunger inside the cylindrical tube is pushed, it moves inside the cylindrical body of the syringe and advances along the printed scale from an initial position to an end position, when the force exerted on the plunger ceases and it is possible to see the dosage taken from the syringe.

Other known dosing systems are commonly known as pistol-types, in which the tube to be emptied is connected to a plunger that is moved by the force exerted on a trigger. Another known dosage form is that of infusion pumps, which is a complex automatic system for the infusion of medication.

Unfortunately, these manual devices are usually not compatible with different types of general syringes or plunger containers and are often complex, high-cost systems. In addition, their accuracy would need to be improved to make all the doses the same without the need for another type of additional measurement.

The purpose of this invention is to provide a dosing device which allows a dose of compound to be given manually and accurately and which is compatible with various types of syringes. At the same time, it is desirable for the device to be disposable, i.e., single use, so that it can be manufactured at low cost.

### BRIEF DESCRIPTION OF THE INVENTION

The present invention has the object of a dosing device for blood compounds or medical compounds, which can be used manually and can extract several exact doses of compound.

The dosing device comprises a stepped body and a presser mechanism. The dosing device can be attached to various different types of syringe with a simple change to its diameter in each case. The presser mechanism is attached to the proximal end of a syringe tube, which has an inner cavity. The syringe plunger comprises a stepped body which are movable along the inner cavity of the syringe tube. The plunger has a piston at the distal end, which is in tight contact with the tube wall, so that, when the plunger moves together with the stepped body, they exert a force on the piston and the piston on the fluid that is in the inner cavity of the tube. In this way, the fluid is displaced from the proximal end of the tube to the outside of the tube through the distal end of the tube.

The stepped body comprises an elongated body, at least one stepped area, with longitudinally consecutive steps along this elongated body. Being the distance between the steps is what in turn determines the amount of a dose.

The presser mechanism which is attached to the open proximal end of the tube, comprises a U-shaped body and a presser connected to the body by one of its ends, where the presser comprises a projection towards the inside of the presser mechanism.

Once the dosing device is mounted on a syringe, the presser mechanism is in contact with the stepped body: specifically, the projection of the presser is in contact with a step on the stepped body. The projection exerts a force on the step on the stepped body of the plunger and lowers it with respect to the proximal end of the tube, so that the piston of the distal part of the plunger exerts a directly proportional force on the fluid compound that is within the inner tube cavity and causes it to move out of the tube through the distal end at the required dose.

The device of this invention, allows extracting from a syringe, an exact dose of compound in a simple and convenient way, since the dosing device can be attached to almost any type of syringe with a simple adaptation of diameter. The device can be used easily with just one hand. In addition, the dosing device has a low manufacturing cost so that it can be disposable.

### BRIEF DESCRIPTION OF DRAWINGS

The details of the invention are shown in the following figures, which do not intend to limit the scope of the invention:

FIG. 1 shows a perspective view of the dosing device fitted into a normal syringe.

FIG. 2 shows a perspective view of the preferred embodiment, where the stepped body is manufactured together with the plunger as one part.

FIG. 3 shows a perspective view of the presser mechanism of FIG. 1.

FIG. 4 shows an elevation view of the presser mechanism of FIG. 3.

FIG. 5 shows a perspective view of the presser mechanism of FIG. 3 fixed onto the tube of FIG. 1.

FIG. 6 shows a cross-sectional view of the device showing the sequence of extraction of a dose.

FIG. 7 shows a perspective view of an alternative embodiment of the stepped body of FIG. 2.

FIG. 8 shows a side-on view of the stepped body of FIG. 7.

FIG. 9 shows a perspective view of the alternative embodiment of FIG. 7 attached to the plunger.

FIG. 10 shows an alternative embodiment, in which the plunger has two different stepped bodies.

FIG. 11 shows an alternative embodiment, in which the steps of the stepped body are positioned in the inverse direction.

### DETAILED DESCRIPTION OF THE INVENTION

The invention relates to a dosing device (1), capable of delivering a specific dose (d) in a precise way without the need for a complex additional measuring system. The dosing device (1) comprises a stepped body (9) and a presser mechanism (17), which are attached to a tube (2) and a plunger (3) of a syringe, thus forming a manual dosing device (1) according to the invention.

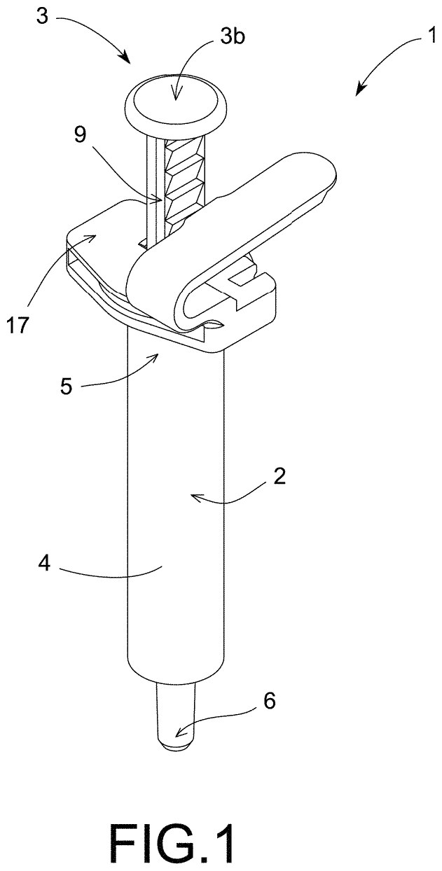

FIGS. 1 to 6 show the preferred embodiment of the dosing device (1) of the invention. FIG. 1 shows the perspective view of the dosing device (1) with all its components on a syringe.

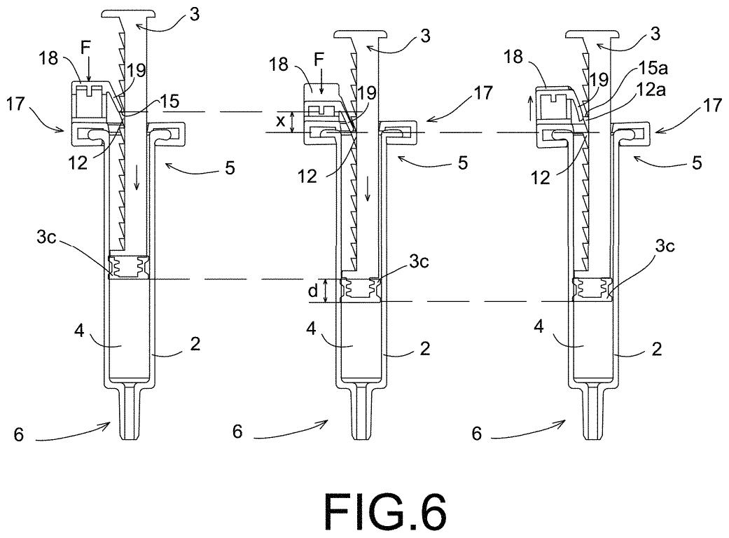

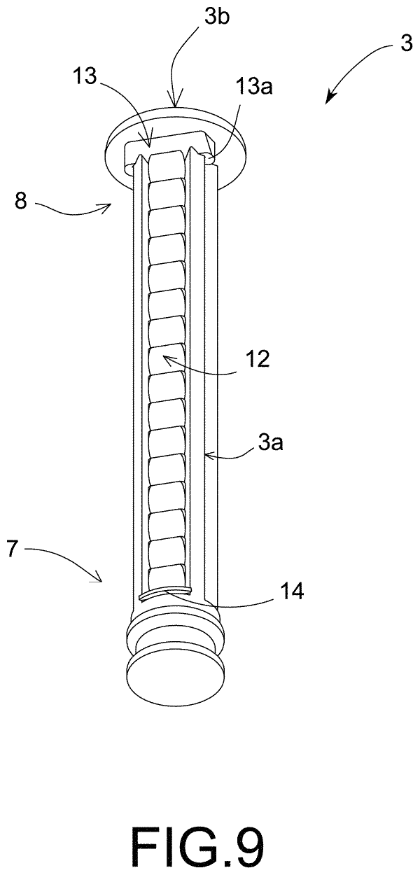

In general, conventional syringes comprise a tube (2) with an inner cavity (4), which extends from an open proximal end (5) of the tube (2) to an open distal end (6) of the tube (2). Conventional syringes further comprise a plunger (3) which can be moved along the inner cavity (4) of the tube (2). The plunger (3) of conventional syringes is a body with triangular grooves (3a) as seen in FIG. 9; a flat head (3b) at the proximal end (8) of the plunger (3), where the force (F) is applied for the extraction of a dose (d); and a piston (3c) at the distal end (7) of the plunger (3). The piston (3c) is in tight contact with the side wall of the tube (2), as shown in FIG. 6, so that when the plunger (3) moves a distance (x) due to force (F), the piston (3c) exerts a force proportional to the force (F) directly on a fluid of the inner cavity (4) of the tube (2), whereby the fluid is displaced the same distance (x) as the piston (3c) towards the open distal end (6) of the tube (2) until the cessation of the force (F).

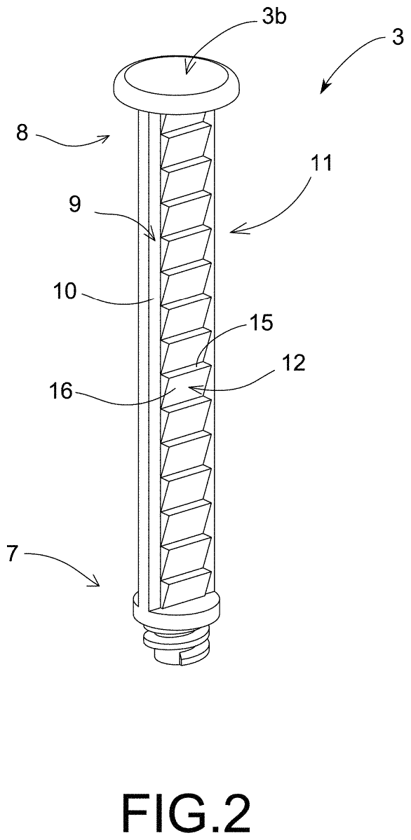

On one hand, the plunger (3) of the invention comprises a stepped body (9), which can be seen in more detail in FIG. 2. The stepped body (9) comprises an elongated body (10) which can be manufactured attached to the plunger (3). This elongated body (10) comprises a stepped area (11) with longitudinally consecutive steps (12) along the elongated body (10). The steps (12) are preferably triangular and equidistant and comprise an upper part or flat area (15) and a lower part or ramp (16).

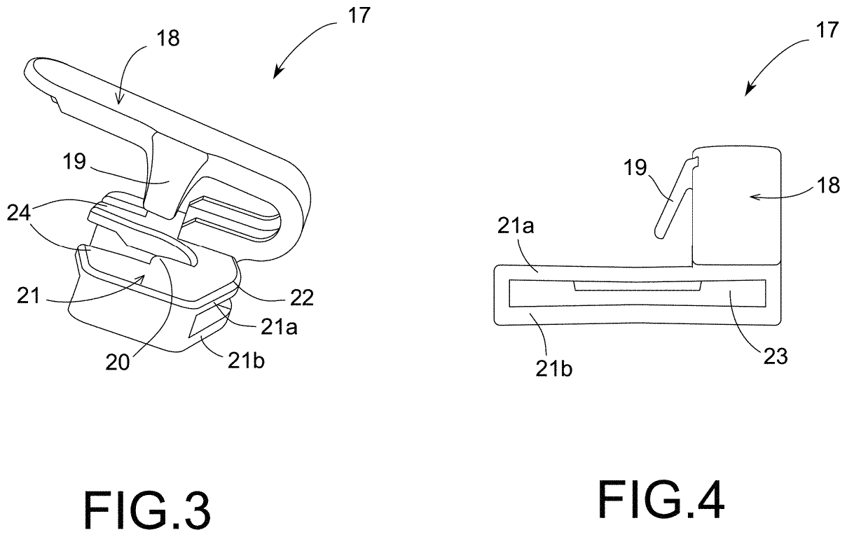

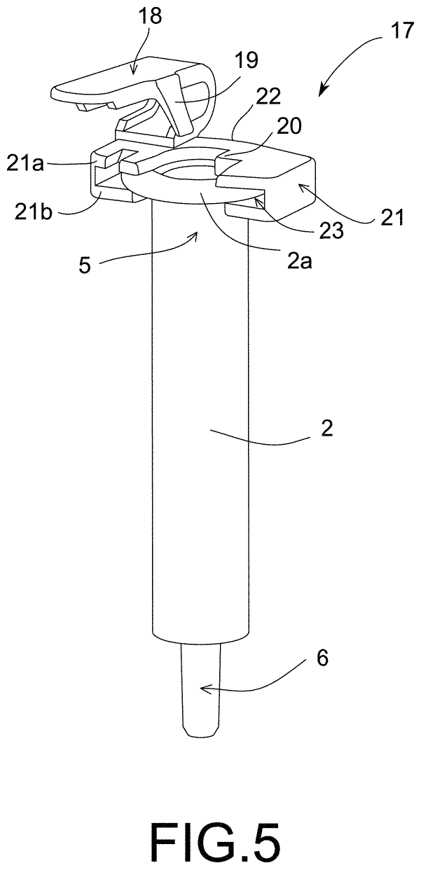

On the other hand, in FIGS. 3 and 4 is shown the presser mechanism (17), and it is attached to the open proximal end (5) of the tube (2) of the invention, as seen in FIG. 5. The presser mechanism (17) comprises a U-shaped body (21), formed of at least two superimposed layers (21a, 21b) in the same U-shape as the body (21). The body (21) comprises an area called base (22), a wedge (20) and arms (24). On the upper layer (21a), a pusher (18) extends from the base (22) upwards in a C-shape, from which a projection (19) sticks out in a distal direction towards the interior of the body (21), as shown in FIGS. 3 and 4. In addition, on the upper layer (21a) there is also a wedge (20) intended to stop the rotation of the plunger (3) within the tube (2), by means of inserting the wedge (20) into a groove (3a) of the plunger (3). The layers (21a, 21b) are perpendicularly joined by the outer areas of the arms (24) of the body (21). Between the layers (21a, 21b) there is a gap (23), as seen in FIG. 4.

In FIG. 5, can be seen how is the join between the presser mechanism (17) and the tube (2). The open proximal end (5) of the tube (2) is housed inside the body (21); more specifically, the rim (2a) of the tube (2) of the conventional syringe is housed in the gap (23) between the layers (21a, 21b). This join can also be made in other ways; for example, with a screw system if the syringe requires it.

When the presser mechanism (17) is joined to the proximal open end (5) of the tube (2), the wedge (20) of the presser mechanism (17) faces towards the inner cavity (4) so that when the plunger (3) with the stepped body (9) in the tube (2) is inserted, this wedge (20) fits into one of the grooves (3a) of the plunger (3) impeding the plunger (3) from turning inside the tube (2). This arrangement of the wedge (20) means that the projection (19) of the presser mechanism (17) always faces the step (12) of the stepped area (11) of the stepped body (9), making the dosing device (1) ready for dosage.

FIG. 6 shows the preferred dose (d) sequence according to the invention. The dosing device (1) is based on the realization of a simple dosage of a specific dose (d), when the user presses the pusher (18) with the finger and exerts a force (F) in a downward direction on the presser mechanism (17). By exerting the force (F) on the pusher (18), the projection (19) which is connected to the pusher (18) and is located on the upper flat area (15) of the step (12), is also subjected to the force (F). Thus, the projection (19) pushes the step (12) of the stepped body (9) until the cessation of force (F) or to the maximum displacement, at which point the projection (19) and the step (12) cease to be in contact. When the step (12) moves down, the stepped body (9) and the plunger (3) are pushed together from an initial position to an end position through the inside of the tube (2). In the initial position, the projection (19) is in the upper flat area (15) of the step (12), and by exerting a downward force (F) on the pusher (18), the step (12) moves downwards a distance (x) with respect to the presser mechanism (17), until the pusher (18) cannot push down or deform further, the projection (19) ceases to be in contact with the step (12) and the advance of the plunger (3) stops in its final position. Then, the user stops exerting force (F), and the projection (19) and the pusher (18) tend to return to their initial position which, after dosing, is the upper part or flat area (15a) of the upper step (12a). So that a new sequence can be started, for a new dose (d). This dosing system allows the user to provide treatments or compounds in exact quantities or doses (d) without effort or additional measurements, simply by pressing the pusher (18) once or more. To make the dosage possible, the dosing device (1) is composed of a flexible, elastic material, such as plastic, which allows the pusher (18) and the projection (19) to deform and then return to their initial positions.

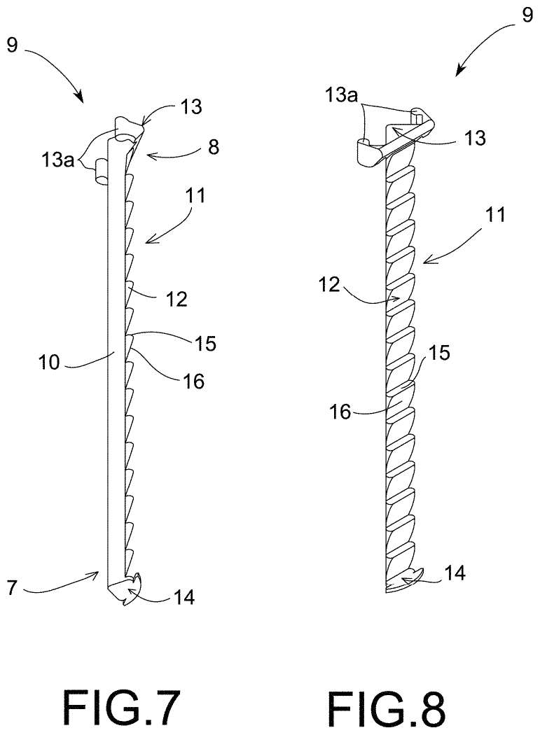

In alternative embodiments it is contemplated that the stepped body (9) can be attachable. i.e. it can easily be attached and detached from the plunger (3). To do so, the stepped body (9) must have at least one upper or lower clipping system (13, 14), which connects the stepped body (9) to the plunger (3), as seen in FIGS. 7 to 9.

FIG. 9 shows the connection of the plunger (3) with the stepped body (9) by means of the clipping system (13, 14). In this case, the elongated body (10) of the stepped body (9) is inserted into a groove (3a) on the plunger (3), thus being hidden in the groove (3a). The join is made at the proximal end (8), by means of arms (13a), which form the upper clipping system (13) seen in FIGS. 7 and 8. The arms (13a) exert a small pressure, and therefore the stepped body (9) is firmly fixed to the plunger (3) so that it moves together with the plunger (3) through the inner cavity (4) of the tube (2). The fact that the stepped body (9) is attachable makes the dosing device (1) very versatile, since it can be attached to different types of plungers (3), simply by modifying the size or shape of the elongated body (10) so that it matches the shape of the groove (3a) in the plunger (3).

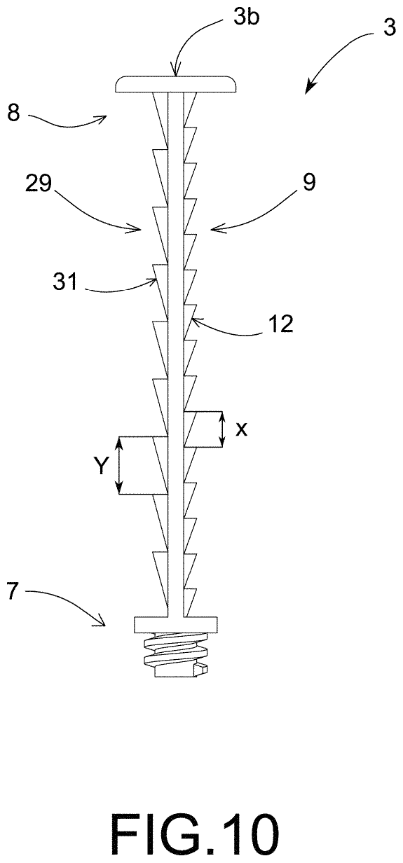

It is also contemplated, in alternative embodiments, that the steps (12) do not have to be triangular and may have some other shape. It is also contemplated that the dosing device (1) may be manufactured with two or more different stepped bodies (9, 29) in the plunger (3), as seen in FIG. 10. The stepped bodies (9, 29) have two bodies (10, 30) which are elongated and stepped in different ways and have steps (12, 31) at different distances (x, Y), so that with a simple turn of the plunger (3) with respect to the tube (2), they apply or supply the dose (d, D) of different specific amounts. The rotation of the plunger (3) involves detaching the presser mechanism (17) from the tube (2), then turn the plunger (3) towards the dosage desired and reattaching the presser mechanism (17), so that the projection (19) faces the step (12, 31) of the desired dose (d, D). The advantage of having several different dosages in the same dosing device (1) is the versatility that allows you to apply different doses without changing from one device to another.

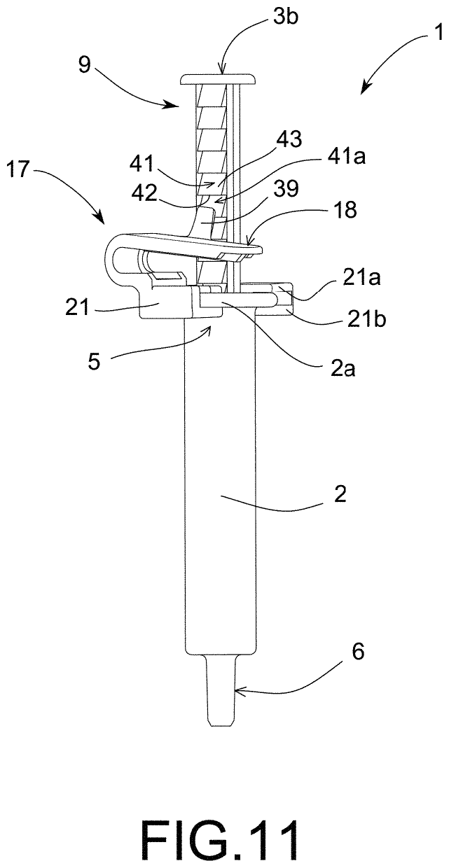

As shown in the FIG. 11, a different embodiment of the dosing device (1) is based on a stepped body (9) which comprises an elongated stepped body (10) which is inverse stepped to the previous alternatives with steps (41); i.e., the step (41) has a flat area (42) in the lower part of the step (41) and a ramp (43) in the upper part of the step (41), so that instead of supplying fluid to another device, the dosing pulls the fluid into the inner cavity (4) of the tube (2) of the dosing device (1). In this case, the projection (19) of the presser mechanism (17) is located in the pusher (18), in the inner part of the body (21), but in a proximal direction, instead of distal direction as in the previous alternatives. As interpreted in FIG. 11, the initial position of the projection (19) is in contact with the flat area (42) of the step (41). In this way, the user exerts a downward force (F) on the pusher (18) until the projection (19) returns to meet the flat area (42) of the next step (41a), which is the moment at which the force (F) ceases. When the force (F) ceases, the pusher (18) tends to return to its initial position, in the upward direction, so that it pushes the next step (41a) in a proximal direction a distance (x), thereby pulling the plunger (3) the same distance (x), which will be proportional to the dose (d) that is introduced into the inner cavity (4) of the tube (2). This system is very useful when extracting certain substances from some other device, such as a plasma fraction from a complete blood sample.

This last alternative would also be possible when the stepped body (9) was attachable and detachable from the plunger (3) or if there were 2 stepped bodies (9, 29) with steps (41) of different sizes, manufactured directly onto the plunger (3).

Finally, the design of the dosing device (1) should be adapted slightly so that it is compatible with each brand and thickness of syringes. However, it is also possible that the dosing device (1) may include the tube (2) and the plunger (3).

1. Dosing device (1) which is characterised in that it comprises:
a stepped body (9), with an elongated body (10), a stepped area (11), which comprises a longitudinally consecutive steps (12) along the elongated body (10);
a presser mechanism (17), comprising a body (21) from which a pusher (18) extends, which comprises a projection (19), which pushes the step (12) of the stepped body (9) a certain distance (x).

2. Dosing device (1) according to claim 1, characterized in that the stepped body (9) is manufactured directly on a plunger (3).

3. Dosing device (1) according to claim 1, characterized in that the stepped body (9) is connectable and detachable from a plunger (3).

4. Dosing device (1) according to claim 1, characterized in that the steps (12) are equidistant.

5. Dosing device (1) according to claim 1, characterized in that the steps (12) comprise an upper part or flat area (15) and a lower part or ramp (16).

6. Dosing device (1) according to claim 1, characterized in that the presser mechanism (17) is connectable and detachable from a tube (2).

7. Dosing device (1) according to claim 1, characterized in that the body (21) of the presser mechanism (17) is U-shaped.

8. Dosing device (1) according to claim 1, characterized in that the body (21) is formed of at least two substantially parallel, superimposed layers (21a, 21b).

9. Dosing device (1) according to claim 1, characterized in that the body (21) comprises a wedge (20).

10. Dosing device (1) according to claim 1, characterized in that the presser mechanism (17) comprises a gap (23) between the layers (21a, 21b), for fitting the rim (2a) of the tube (2).

11. Dosing device (1) according to claim 3, characterized in that the stepped body (9) has at least one clipping system (13, 14) for joining it to the plunger (3).

12. Dosing device (1) according to claim 11, characterized in that the clipping system (13) comprises arms (13a).

13. Dosing device (1) according to claim 11, characterized in that there is a clipping system (13, 14) on at least one of the ends of the stepped body (9).

14. Dosing device (1) according to claim 3, characterized in that the elongated body (10) has the form of a groove (3a) on the plunger (3).

15. Dosing device (1) according to claim 2, characterized in that the plunger (3) comprises two or more elongated bodies (10) stepped differently, with the steps (12) at different equidistant distances (x, Y), so that they define two or more different predetermined doses (d, D).

16. Dosing device (1), according to claim 1, characterized in that the stepped body (9) comprises steps (41) which are inverse to the steps (12), where the steps (41) comprise a flat area (42) in the lower part of the step (41) and a ramp (43) in the upper part of the step (41).

17. Dosing device (1) according to claim 1, characterized in that the dosing device (1) further comprises the tube (2) and the plunger (3).
